(19) United States
(12) Patent Application Publication
(10) Pub. No.: US 2008/0221629 A1
(43) Pub. Date: Sep. 11, 2008

(54) Lamination of Lithium Battery Elements for Implantable Medical Devices

(75) Inventors: Adam J. Morgan, Richfield, MN (US); Daniel J. Foster, Fridley, MN (US); Brian L. Schmidt, White Bear Lake, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 11/684,317

(22) Filed: Mar. 9, 2007

(51) Int. Cl. A61N 1/02

(52) U.S. Cl. 607/2; 29/2; 29/623.1; 29/731

(57) ABSTRACT

A method includes the step of providing a sheet of lithium and a sheet of substrate material. The method further includes the step of pressing the sheet of lithium and the sheet of substrate material together in a die, the die having at least one surface that includes a plurality of force concentrating features configured to create regions of relatively higher pressure and regions of relatively lower pressure in at least one of the sheet of lithium and the sheet of substrate material. In another embodiment, a method includes the steps of providing a sheet of lithium material and a substrate material, and applying force to the sheet of lithium and the sheet of substrate material to form a plurality of protrusions on at least one of the sheet of lithium and the sheet of substrate material. The method includes the further step of pressing at least a portion of the sheet of lithium to the substrate material with sufficient force to at least partially deform the protrusions.

### FIELD OF THE INVENTION

The invention relates to lithium batteries, and more particularly, to lithium batteries for use in implantable medical devices.

### BACKGROUND OF THE INVENTION

A variety of implantable medical devices are used to provide medical therapy to patients. One example of a type of implantable medical devices is a cardiac rhythm management (CRM) device. CRM devices may include, for example, pacemakers and implantable cardioverter defibrillators (ICD). These devices generally function to provide treatment to a patient having a disorder relating to the pacing of the heart, such as bradycardia or tachycardia. For example, a patient having bradycardia may be fitted with a pacemaker, where the pacemaker is configured to monitor the patient's heart rate and to provide an electrical pacing pulse to the cardiac tissue if the heart fails to naturally produce a heart beat in a given time interval. By way of further example, a patient may have an ICD implanted to provide an electrical shock to the patient's heart if the patient experiences atrial fibrillation.

These implantable medical devices require power to operate and this power is typically provided by a battery within the device. Battery performance is a key aspect of the performance of the device. Long battery life is very important because battery replacement generally involves surgery, which is inconvenient for the patient, expensive, and exposes the patient to the risk of complications. Battery reliability is also important because of the critical nature of functions that may be performed by an implanted medical device.

Implantable medical devices commonly use lithium-based batteries. Lithium batteries have a number of advantageous characteristics that make them desirable for use in implantable medical devices. Lithium batteries can be formed into a wide variety of shapes and sizes to efficiently make use of the space available within the implantable device. Lithium batteries also have high power to weight ratios and high power density, which allows for smaller battery size and longer battery life for a given size.

The power output requirements of batteries used in implantable medical devices depend on the type of medical device. For example, batteries used in an ICD have both a low rate requirement and a high rate requirement. The battery must provide continuous low rate current to supply power control electronics and to provide low output therapy in the form of cardiac pacing pulses. The battery must also provide occasional high current pulses to charge high voltage capacitors, which in turn are used to defibrillate the patient's heart. Often more than one pulse is required to defibrillate a patient requiring, thus the battery is capable of delivering multiple high current pulses in a quick succession.

It is important that a lithium battery for use in an implantable medical device be able to satisfy the device power requirements for as long of a period as possible. However, the techniques used to construct the battery can have a significant influence on the performance of the battery and the battery's ability to satisfy the power requirements. For example, in an embodiment of a battery where a lithium sheet is laminated to a current collector, the performance of the battery depends significantly on the quality and durability of the contact between the lithium element and the current collector. Battery performance will be degraded if the contact between the lithium and the current collector is poor. Improved techniques for laminating lithium sheets are needed.

### SUMMARY OF THE INVENTION

One aspect of the invention relates to a method including the steps of providing a sheet of lithium and a sheet of substrate material and pressing the sheet of lithium and the sheet of substrate material together in a die to form an electrode, where the die has at least one surface that includes a plurality of force concentrating features configured to create regions of relatively higher pressure and regions of relatively lower pressure in at least one of the sheet of lithium and the sheet of substrate material. The method further includes the steps of assembling a battery including the electrode in a battery housing and assembling an implantable medical device including the battery housing.

A further aspect of the invention also relates to a method including the steps of providing a sheet of lithium material and a substrate material and applying force to the sheet of lithium and substrate material to form a plurality of protrusions on at least one of the sheet of lithium and the substrate material. The method further includes the step of pressing at least a portion of the sheet of lithium to the substrate material with sufficient force to at least partially deform the protrusions and form an electrode. The method further includes the steps of assembling a battery including the electrode in a battery housing and assembling an implantable medical device including the battery housing.

Another aspect of the invention relates to a tool for processing a sheet for use in an electrode of a battery. The tool includes a first rigid surface that has a plurality of force concentrating features. This first rigid surface is configured to engage with a surface of the sheet. The tool further includes a second rigid and generally smooth surface that is positioned opposite to the first rigid surface. The tool also includes a mechanism to force the first and second surfaces together against at least the sheet with sufficient force that the plurality of force concentrating features will at least partially deform the sheet.

Yet another aspect of the invention relates to a tool for processing a sheet for use in an electrode of a battery. The tool includes a first rigid surface that has a plurality of protrusions, where the first rigid surface is configured to engage with a surface of the sheet, and a second surface, where the second surface has one or more openings that are configured to provide clearance opposite to the protrusions of the first rigid surface when the first and second surfaces are pressed together. This second surface is configured to engage with at least a portion of the second surface of the sheet. The tool also includes a mechanism to force the first and second surfaces against the sheet with sufficient force to cause the plurality of protrusions to deform the sheet.

An additional aspect of the invention relates to an implantable medical device including a housing, at least one lead, and circuitry configured to send and receive electrical impulses through the lead. The implantable medical device further includes a lithium battery configured to provide electrical power to the circuitry. The lithium battery includes an electrode having a lamination formed from at least a sheet of lithium and a sheet of a substrate material. The lamination has either a plurality of partially compressed protrusions on the sheet of lithium or a plurality of projections of the sheet of lithium into the surface of the sheet of substrate material.

The invention may be more completely understood by considering the detailed description of various embodiments of the invention that follows in connection with the accompanying drawings.

### BRIEF DESCRIPTION OF THE DRAWINGS

FIG. 1 is an exploded view of a lithium battery.

FIG. 2 is a cross-sectional view of a repeating assembly of the lithium battery of FIG. 1.

FIG. 3 is a partial exploded view of a cathode stack electrically connected to a pin that extends through a case of a lithium battery.

FIG. 4 is a perspective view of a die assembly constructed according to the principles of the present invention.

FIG. 5 is a perspective view of a die block of the die assembly of FIG. 4.

FIG. 6 is a partial cross-sectional view of an embodiment of depressions of the die block of FIG. 6.

FIG. 7 is a partial cross-sectional view of an alternative embodiment of depressions of the die block of FIG. 6.

FIG. 8 is a partial cross-sectional view of an alternative embodiment of depressions of the die block of FIG. 6.

FIG. 9 is a partial cross-sectional view of an embodiment of protrusions of the die block of FIG. 6.

FIG. 10 is a partial cross-sectional view of an alternative embodiment of protrusions of the die block of FIG. 6.

FIG. 11 is a partial cross-sectional view of an alternative embodiment of protrusions of the die block of FIG. 6.

FIG. 12 is a perspective view of an alternative embodiment of a die block.

FIG. 13 is a partial cross-sectional view of an embodiment of a groove of the die block of FIG. 12.

FIG. 14 is a partial cross-sectional view of an alternative embodiment of a groove of the die block of FIG. 12.

FIG. 15 is a partial cross-sectional view of an alternative embodiment of a groove of the die block of FIG. 12.

FIG. 16 is a partial cross-sectional view of an embodiment of a ridge of the die block of FIG. 12.

FIG. 17 is a partial cross-sectional view of an alternative embodiment of a ridge of the die block of FIG. 12.

FIG. 18 is a partial cross-sectional view of an alternative embodiment of a ridge of the die block of FIG. 12.

FIG. 19 is a schematic cross-sectional view of the die assembly of FIG. 4.

FIG. 20 is a schematic of a die assembly for post-processing an anode assembly.

FIG. 21 is a schematic view of a die assembly for preprocessing a lithium sheet.

FIG. 22 is a schematic view of an alternative die assembly for preprocessing a lithium sheet.

FIG. 23 is an alternative embodiment of a die assembly for preprocessing a lithium sheet.

FIG. 24 is a schematic view of a die assembly for forming a lamination of a current collector to a lithium sheet that has been preprocessed with the die assembly of FIG. 20.

FIG. 25 is a schematic view of an implantable medical device and a heart.

While the invention may be modified in many ways, specifics have been shown by way of example in the drawings and will be described in detail. It should be understood, however, that the intention is not to limit the invention to the particular embodiments described. On the contrary, the intention is to cover all modifications, equivalents, and alternatives following within the scope and spirit of the invention as defined by the claims.

### DETAILED DESCRIPTION OF THE INVENTION

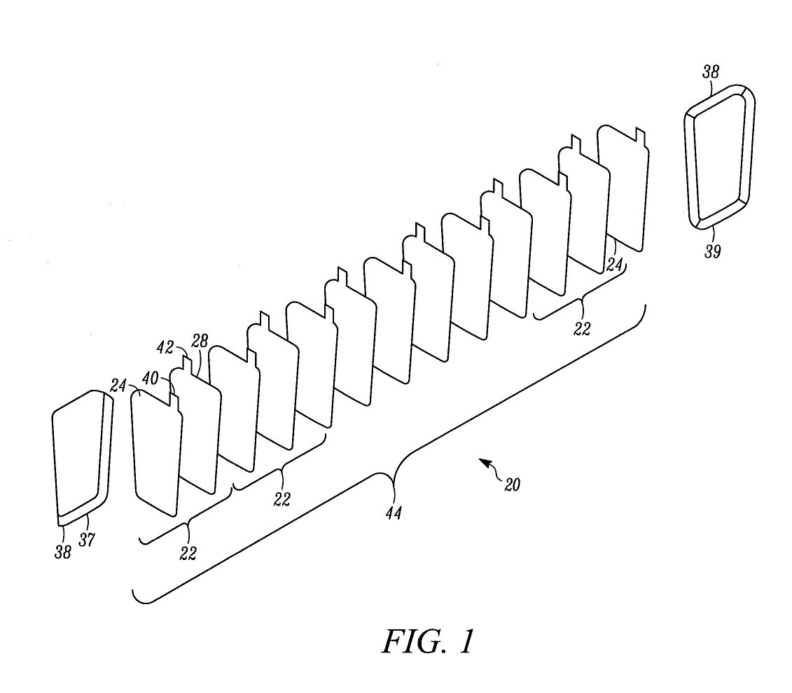

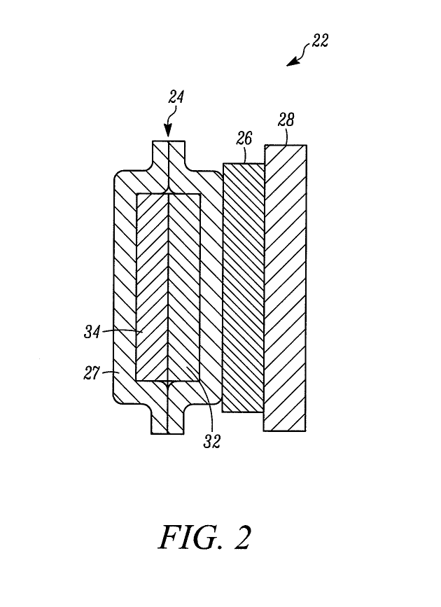

One typical lithium battery type for an implantable medical device is a lithium-manganese dioxide (Li/MnO₂) battery. However, other lithium battery chemistries are also usable. Lithium batteries are typically constructed from a number of thin sheets of different materials that are sandwiched together to form a battery assembly. An example embodiment of a lithium battery constructed in this way is shown in FIG. 1, which for clarity only depicts certain elements of the lithium battery. In the embodiment of FIG. 1, battery assembly 20 is formed from a repeating arrangement 22 of elements. A cross-section of the repeating arrangement 22 is depicted in FIG. 2. Repeating arrangement 22 includes an anode assembly 24, a separator 26, and a cathode 28.

The anode assembly 24 is formed from a sheet of lithium material that constitutes an anode 32 and a material that constitutes a current collector 34. Various embodiments of the application include methods and tools for laminating a sheet of lithium material to a substrate, such as a current collector. Another embodiment of the invention is an implantable medical device including a lithium battery that has an electrode constructed according to the present invention. The current collector may be constructed from a number of different materials. For example, the current collector 34 may be constructed from, among other alternatives, nickel or nickel-based material, stainless steel, aluminum, titanium, or copper. The current collector 34 may comprise a uniform sheet, a wire grid, or other configurations. For convenience of description, the current collector 34 will be referred to below as being constructed from a sheet of nickel or nickel-based material, but it will be recognized that the current collector 34 may alternatively be constructed from any suitable material or configuration without departing from the principles disclosed herein.

The anode assemblies 24 are constructed by pressing together a lithium anode 32 to a nickel current collector 34. The process of pressing the lithium anode 32 to the nickel current collector 34 may also be called lamination.

The elements of repeating arrangement 22 are generally constructed in a shape that is configured to suit the space available in the implantable medical device. Each of the elements of repeating arrangement 22 generally has the same shape. However, one important aspect of repeating arrangement 22 is the need to prevent direct contact between the cathode 28 and the anode 24. If direct contact were to occur, rapid electrical discharge would occur with significant electrical current flowing between the components. This situation could impair the operation of the battery. For this reason, it is important to ensure that the cathode 28 will not contact the anode 24. However, in some embodiments, the cathode 28 is formed from a die cut metal grid that has been coated with a slurry mixture. For these embodiments, the edges of this die cut metal grid can be very sharp and may be able to penetrate the separator 26 or 30, allowing direct contact between the anode 24 and cathode 28. To minimize the likelihood of this happening, the cathode 28 is typically constructed to be slightly larger than the anode 24, to ensure that the cathode 28 edges are not proximate to the anode 24 and therefore not prone to contact the anode 24.

The lithium anode 32 is preferably constructed to be in complete, firm contact with the nickel current collector 34. Operation of the battery requires that electrons transfer between the lithium anode 32 and the nickel current collector 34. However, this can only occur at locations where the anode 32 is actually in contact with the current collector 34. If there are regions of the anode 32 and current collector 34 that are not actually in contact, even though they may be very close to each other, current will either not be able to transfer at such locations or the resistance to current will be high. The presence of locations where current cannot transfer or is subject to high resistance will undesirably limit the performance of the battery. For example, the rate of discharge capabilities of the battery may be degraded. This effect may be especially noticeable near the end of battery life. For at least these reasons, the lithium anode 32 and the nickel current collector 34 are preferably brought together in a fashion that promotes complete, firm contact between them. A lamination process is typically used to create the contact between the lithium anode 32 and the nickel current collector 34.

A further issue exists with respect to the contact between the anode 32 and the current collector 34. Namely, lithium has a tendency to form a layer that consists of lithium byproducts on its surface. Not wanting to be bound by theory, it is believed that lithium reacts with certain materials that it comes in contact with, such as gases within air, materials in processing and manufacturing equipment, and materials in storage or handling equipment. For example, it is believed that lithium reacts with atmospheric N₂ and CO₂ to form byproducts on its surface. When the lithium anode 32 is pressed against the current collector 34, a layer of lithium byproducts can inhibit the flow of electrons from the anode 32 to the current collector 34. It is therefore desired that the contact between the lithium anode 32 and the current collector 34 be directly against a fresh lithium surface without having byproducts at the contact surface.

A lamination of anode 32 and current collector 34 is typically created by applying pressure to the lithium anode 32 and the nickel current collector 34 to force them together. An issue arises, however, because lithium is very malleable. As pressure is applied to the lithium anode 32, it has a tendency to extrude and expand outward. This extrusion outward can cause the lithium anode 32 to approach, or equal or exceed, the size of the cathode 28 that is constructed to be nominally larger than the lithium anode 32. In some cases, the amount of pressure that can be applied to the lithium anode 32 without causing excessive extrusion is not sufficient to properly laminate the lithium anode 32 to the current collector 34. If the pressure applied to the lithium anode 32 is increased in an attempt to better laminate it to the current collector 34, the lithium may extrude outward excessively, possibly to a size that would make it more difficult to align the cathode 28 and anode 32 to avoid direct contact, or to a size that would allow direct contact between the cathode 28 and anode 32 regardless of the alignment of the cathode 28 and anode 32 after the battery is assembled. Because higher pressure lamination tends to cause increased extrusion of the anode and consequently reduced accuracy in the anode size, simply increasing the lamination pressure would increase the likelihood of a short circuit.

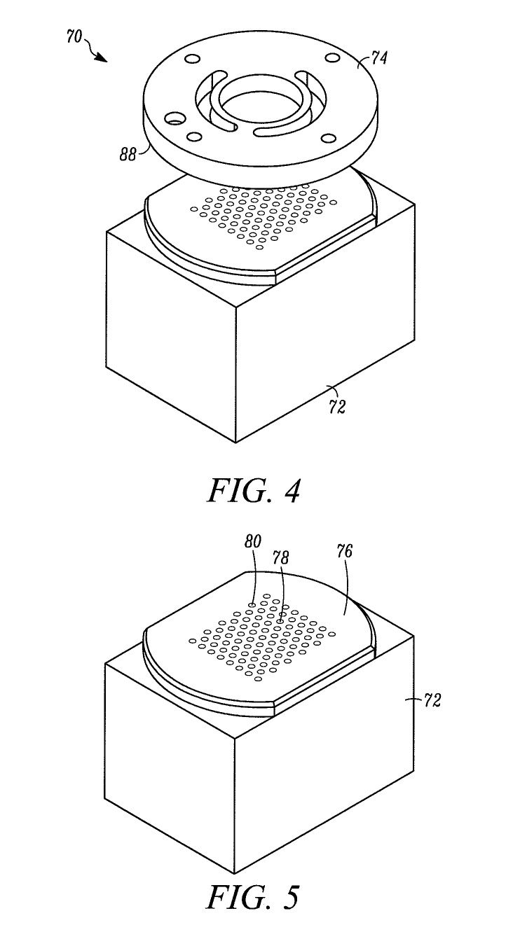

A die assembly configured for advantageously laminating an anode 32 to a current collector 34 is depicted in FIG. 4. Die assembly 70 includes die base 72 and die pressure plate 74. An alternative perspective view of die base 72 is shown in FIG. 5. Die base 72 includes a material support surface 76. Material support surface 76 is sized to at least support the entire surface of an anode 32 and/or a current collector 34. Material support surface 76 includes a plurality of pressure concentrating features 78. Pressure concentrating features 78 may be formed by any of a number of manufacturing processes known to those of skill in the art. For example, pressure concentrating features 78 may be formed by electro-discharge machining (EDM), ablation, chemical etching, machining, etc.

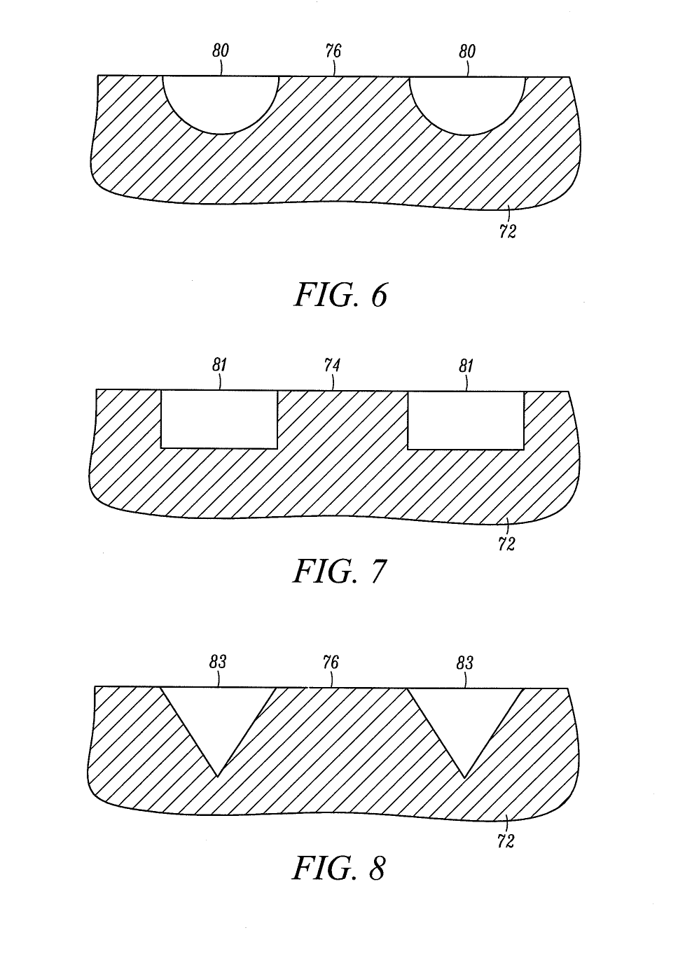

In one embodiment, pressure concentrating features 78 include a plurality of depressions 80 in material support surface 76. These depressions 80 may take any of a number of shapes and configurations. FIGS. 6, 7, and 8 are partial cross-sectional views of different embodiments of depressions (not to scale). For example, in various embodiments depressions 80 are each characterized by a round or semi-spherical (such as in FIG. 6) depression below the material support surface 76. In other embodiments, depressions 81 are square, rectangular, or cylindrical, so that they have the square-type cross section shown in FIG. 7. In other embodiments, depressions 83 are triangular or conical in cross section as shown in FIG. 8. These depressions may even be irregular in cross-section. In certain embodiments, depressions 80 are generally round or semi-spherical and are characterized by a diameter (at the material support surface 76) of about 0.003 inches (0.076 mm) and a depth from material support surface 76 of about 0.003 inches (0.076 mm). In certain embodiments, depressions are characterized by a width or diameter at the material support surface 76 of 0.001 to 0.05 inches (0.025 to 1.27 mm) and a depth of about 0.001 to 0.05 inches (0.025 to 1.27 mm) below material support surface 76.

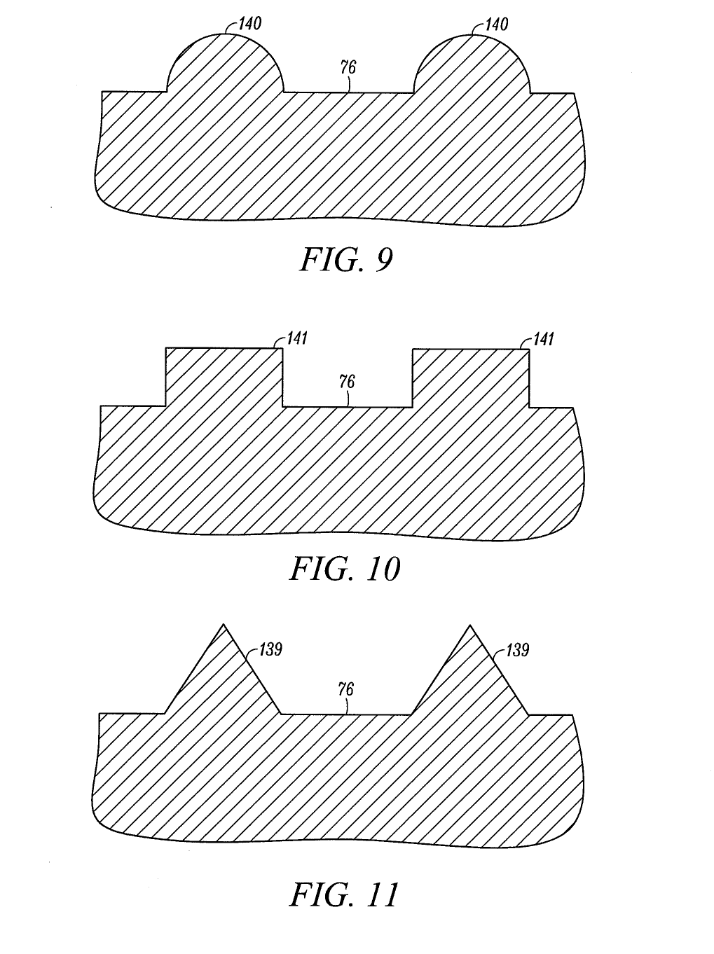

In yet another embodiment, pressure concentrating features 78 include a plurality of embosses or protrusions. These protrusions may take any of a number of shapes and configurations. FIGS. 9, 10, and 11 are partial cross-sectional views of different embodiments of protrusions (not to scale). For example, in various embodiments protrusions are each characterized by a round or semi-spherical protrusion 140 above the material support surface 76 such as in FIG. 9. In certain embodiments, protrusions 141 are square, cylindrical, or rectangular, having a square-type cross-section as in FIG. 10. In other embodiments, the protrusions 139 are triangular or conical as in FIG. 11, or even irregular in cross section. In certain embodiments, protrusions 140 are generally round or semi-spherical and are characterized by a diameter (at the material support surface 76) of about 0.003 inches (0.076 mm) and a height above material support surface 76 of about 0.003 inches (0.076 mm). In certain embodiments, protrusions are characterized by a width or diameter at the material support surface 76 of 0.001 to 0.05 inches (0.025 to 1.27 mm) and a height above material support surface 76 of about 0.001 to 0.05 inches (0.025 to 1.27 mm).

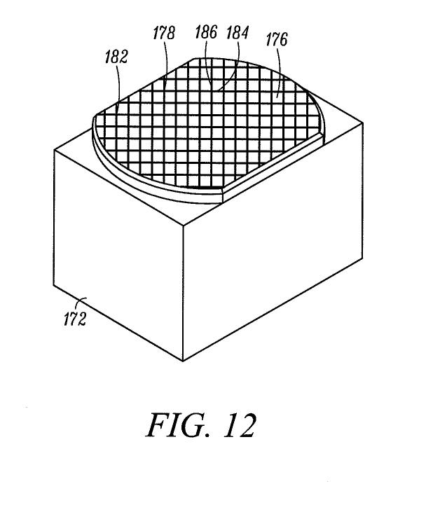

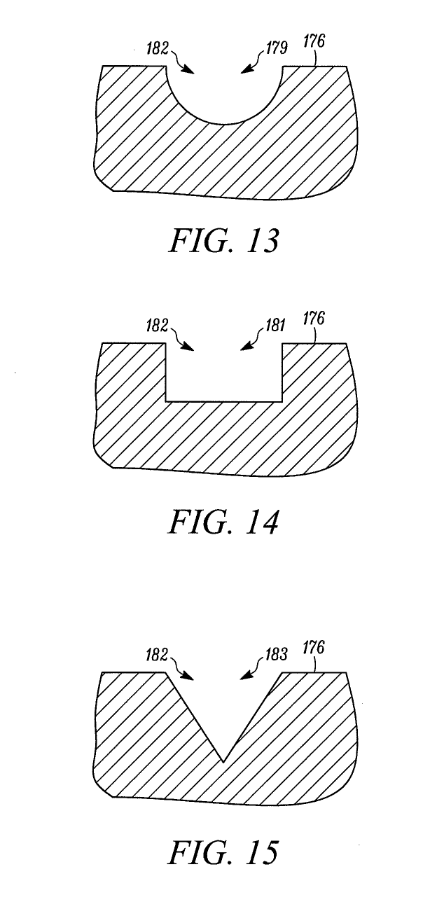

An alternative embodiment of a die base 172 having pressure concentrating features 178 on a material support surface 176 is shown in FIG. 12. In one embodiment, pressure concentrating features 178 are a series of intersecting grooves 182 in material support surface 176. Grooves 182 are shown as being generally in a “waffle board” pattern, having a pattern that is characterized by a first series 184 of spaced apart, parallel grooves that are intersected by a second series 186 of spaced apart, parallel grooves, where the individual grooves of the first series of grooves 184 are each approximately perpendicular to the individual grooves of the second series of grooves 186. Many embodiments of grooves 182 are usable. For example, as shown in FIGS. 13, 14, and 15, respectively, grooves 182 may be characterized by a generally semi-spherical cross-section 179 (as in FIG. 13), a generally square or rectangular cross-section 181 (as in FIG. 14), and a generally triangular cross section 183 (as in FIG. 15). Other cross-sections are usable. In certain embodiments, each groove 182 is about 0.003 inches (0.076 mm) wide and 0.0004 inches (0.010 mm) deep. In other embodiments, each groove 182 is about 0.001 to 0.005 inches (0.025 to 0.127 mm) wide and 0.0001 to 0.005 inches (0.0025 to 0.127 mm) deep. However, many other embodiments of grooves 182 are usable.

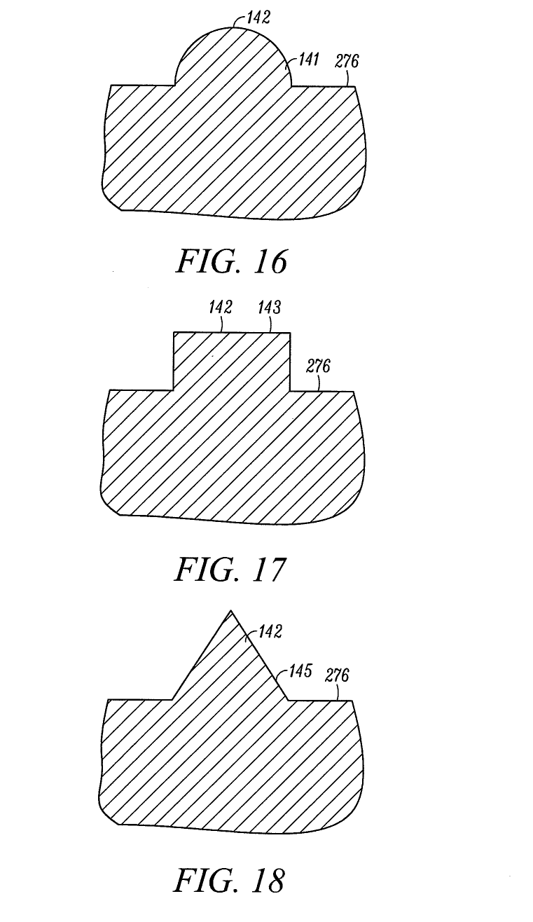

In other embodiments, pressure concentrating features 178 are a series of intersecting ridges 142 in material support surface as shown in FIGS. 16-18. Ridges 142 are constructed and arranged in a manner similar to that of grooves 182, except that ridges protrude above a material support surface 276 in a “waffle board” pattern. Many embodiments of ridges 142 are usable. For example, as shown in FIGS. 16, 17, and 18, respectively, ridges 142 may be characterized by a generally semi-spherical cross-section 141 (as in FIG. 16), a generally square or rectangular cross-section 143 (as in FIG. 17), and a generally triangular cross section 145 (as in FIG. 18). Other cross-sections are also usable. In one embodiment, each ridge 142 is about 0.003 inches (0.076 mm) wide and 0.0004 inches (0.010 mm) high (relative to material support surface 276). In other embodiments, each ridge 142 is about 0.001 to 0.005 inches (0.025 to 0.127 mm) wide and 0.0001 to 0.005 inches (0.0025 to 0.127 mm) high, however, many other embodiments of ridges 142 are usable.

One process of forming an anode assembly from an anode 32 and a nickel current collector 34 will now be discussed with reference to FIGS. 4-5 and 19. The components of die assembly 70 including die base 72 having cylindrical protrusions 141 will be referenced for convenience. However, die base 172 of FIG. 12 or different configurations of die base 72 could be used in a similar manner to accomplish the process. In operation, die assembly 70 is configured to apply pressure to a lithium anode 32 and a nickel current collector 34 to laminate the components together to form anode assembly 24.

Generally, the lithium anode 32 will be in contact with, or facing, the material support surface 76 of die base 72, and the nickel current collector 34 will be in contact with, or facing, the die pressure plate 74. This process will be described in detail, though it is also possible for the current collector 34 to be positioned in contact with, or facing, the material support surface 76 of the die base 72.

In some embodiments, a film may be provided between the die base 72 and lithium anode 32 and between die pressure plate 74 and current collector 34 to minimize sticking of the elements of anode assembly 24 to die assembly 70. In one embodiment, the contact surface 88 of die pressure plate 74 is uniform and generally flat. In another embodiment, contact surface 88 has a plurality of openings that are configured to be subjected to a vacuum. These vacuum openings may be useful for holding the current collector 34 in position with respect to the die pressure plate 74 while the components are being prepared to be laminated. Other surface characteristics or profiles of pressure plate 74 are also usable.

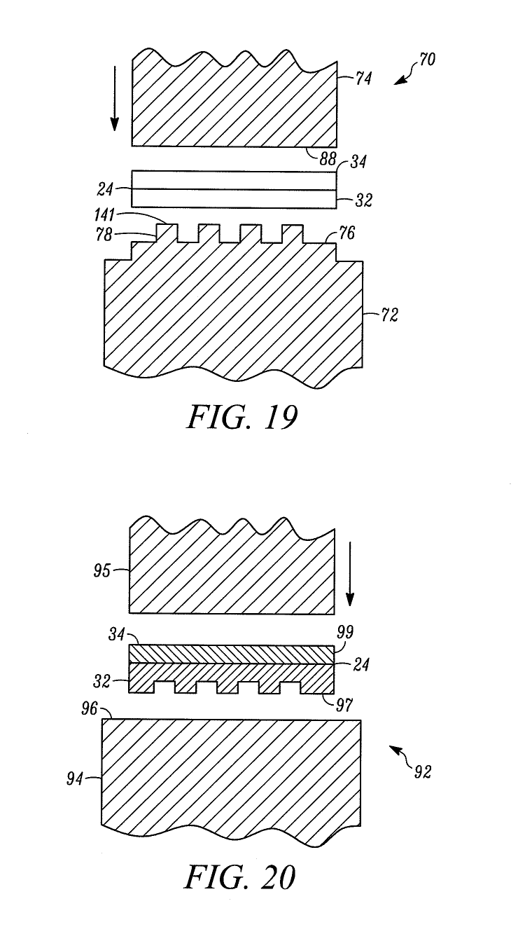

Once the cathode 32 and current collector 34 are positioned within die assembly 70 as shown in FIG. 19, the pressure plate 74 and base 72 are driven towards each other under force, causing pressure to be applied to the anode 32 and current collector 34. The pressure concentrating features 78 cause force to be applied to anode 32 from less than all of material support surface 76. The pressure concentrating features 78 are cylindrical protrusions 141 in FIG. 19, but in other embodiments are depressions, grooves, protrusions, ridges, etc. The pressure concentrating features 78 reduce the surface area in contact with the anode 32, and thus for a given force applied to the die assembly 70, result in greater pressure applied to anode 32 and current collector 34 relative to a die assembly without pressure concentrating features. The regions where pressure is applied to the anode 32 can be called high pressure zones. High pressure zones correspond, in the case where pressure concentrating features 78 are depressions or grooves, to regions of material support surface 76 that are not part of the pressure concentrating features 78, and in the case where pressure concentrating features 78 are protrusions or ridges, to the pressure concentrating features 78 themselves. The greater pressures in the high pressure zones (relative to the lower pressure that would exist with a standard flat die surface that has a larger surface area in contact) promote complete, firm contact between the lithium anode 32 and the nickel current collector 34.

The presence of high pressure zones cause localized pressures within the lithium anode 32 that are greater than the pressures that would be imparted by a standard flat die for the same die assembly force. These greater localized pressures promote complete, firm contact between the lithium anode 32 and the current collector 34, particularly within the high pressure zones. Furthermore, because the high pressure zones exist only in discrete regions of the anode 32 and current collector 34, with the balance of the surface area being under significantly lower pressure, the lithium that extrudes outward under pressure from the high pressure zones will tend to be received by, and expand into, the relief areas defined by the pressure concentrating features 78 such as depressions 80 or grooves 82 or by the space between pressure concentrating features 78 such as protrusions 140 or ridges 142. These relief areas tend to minimize the possibility of the lithium extruding outward under pressure beyond the edges of the lithium sheet. This in turn limits the problem of the anode 32 being prone to contact with the cathode 28.

The existence of high pressure zones has the further advantage of causing microextrusion at the surface of the lithium anode 32. As mentioned, the high pressure zones create areas of higher pressure than exists at the relief areas, and this pressure differential tends to cause the lithium anode 32 material to extrude into the relief areas, such as depressions 80 or grooves 82 or the space between features such as protrusions 140 or ridges 142. This extrusion causes material on the face of lithium anode 32 to be translated laterally, and this lateral translation causes breaks, fissures, and smearing in the surface of the lithium anode 32. These breaks, fissures, and smears provide a fresh lithium surface, which has generally not reacted to form byproducts, to be exposed to the current collector 34. This fresh surface makes more effective and durable electrical contact with the nickel current collector 34 as compared with a surface that includes a layer of byproducts. In some cases, the existence of high pressure zones can cause portions of the lithium anode 32 to project into the surface of the nickel current collector 34.

Now referring to FIG. 20, after anode 32 and current collector 34 are pressed together to form anode assembly 22 using a die assembly 70 having pressure concentrating features, the anode 32 includes a structured surface 97 formed by the pressure concentrating features. This anode assembly 24 having a structured anode surface 97 will be referred to as a structured anode assembly 99. In some embodiments, the structured anode assembly 99 is pressed in a second die assembly 92 to at least partially deform and flatten the structured lithium surface 97. This may be called post-processing of the anode assembly.

Many embodiments of second die assembly 92 are usable. One embodiment is depicted in FIG. 20. In the embodiment of FIG. 20, second die assembly 92 is generally similar to die assembly 70, having a base 94 that has a material support surface 96. However, material support surface 96 does not have pressure concentrating features 78, but instead is generally planar or flat. Second die assembly 92 also includes a pressure plate 95 also having a generally planar or flat surface.

Structured anode assembly 99 is placed within second die assembly 92 and force is applied to the structured anode assembly 99 through base 94 and pressure plate 95. The amount of force applied is generally chosen to be sufficient to at least partially deform and flatten any features on the lithium anode 32 that extend away from the primary plane of the anode 32. Such features may extend away from the primary plane of the anode 32 as a consequence of the uneven application of pressure to the anode 32 through the high pressure zones corresponding to the pressure concentrating features 78. By at least partially deforming and flattening any features that extend from the lithium anode 32, the surface of the lithium anode may be transformed to a surface that is either substantially flat or that has a plurality of partially compressed protrusions or depressions. Applying force to the surface of anode 32 to produce a flatter overall surface of the anode assembly 24 may have several advantages, including denser packaging of the battery elements.

A previously structured surface that has had force applied to at least partially deform and flatten features on the surface may be identified by a number of means, such as through microscopic evaluation of the surface or a cross-section through the lithium sheet for characteristic indications of material flow, folding, or compression. In addition, the surface of a lithium anode 32 that has had compressed protrusions or depressions and has been used in a battery for at least a period of time can show regions of preferential lithium usage, where these regions of greater lithium usage indicate areas that have been in more complete contact with the current collector, such as at compressed protrusions or depressions. Similarly, a surface of the current collector may also be analyzed to detect the presence of indentations or other surface features representative of the existence of regions of high pressure with the lithium sheet. An atomic force microscope or scanning electron microscope can be used to detect these features, for example.

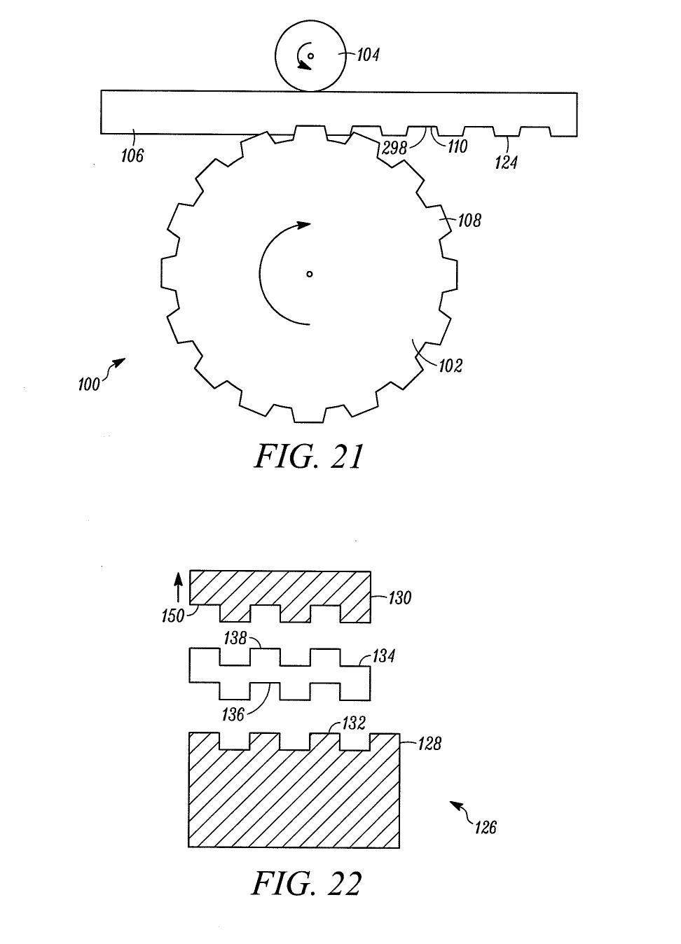

Now referring to FIG. 21, an alternative technique for laminating a lithium anode to a current collector involves forming pressure concentrating features 298 on a lithium anode prior to laminating the anode to a current collector. This technique can be called preprocessing the lithium sheet prior to lamination. Many embodiments for preprocessing a lithium sheet and forming pressure concentrating features 298 on a lithium anode material 106 are usable. One usable embodiment is depicted in FIG. 21. Die assembly 100 includes a first cylindrical roller 102 having a plurality of protrusions 108 around a circumference, and a second cylindrical roller 104 having a generally smooth surface. First cylindrical roller 102 is characterized by a cylinder width and a cylinder diameter.

In one embodiment, protrusions 108 are characterized by ridges that extend laterally across substantially the entire cylinder width and have a square or rectangular or triangular cross section and a height relative to the cylinder of 0.0004 inches (0.010 mm). In other embodiments, protrusions have a height relative to the cylinder of 0.0001 to 0.005 inches (0.0025 to 0.127 mm). In another embodiment, protrusions 108 are characterized by a series of conical protrusions having a height relative to the cylinder of 0.0001 to 0.005 inches (0.0025 to 0.127 mm). The protrusions 108 may have profiles similar to the ridges 142 of FIGS. 16-18, for example.

In operation, first roller 102 is driven, such as by a motor, causing a sheet of lithium 106 to be pulled into the nip formed between first roller 102 and second roller 104. The driving action of lithium sheet 106 causes second roller 104 to rotate. However, the thickness of lithium sheet 106 is generally larger than the shortest distance between first and second rollers 102, 104. Consequently, the protrusions 108 on first roller create corresponding pressure concentrating features 298, such as depressions 110 and protrusions 124, on lithium sheet 106 after it has been pulled through the nip of the rollers 102, 104. The shape of the pressure concentrating features 298 will generally correspond to the shape of the protrusions, such that, for example, a series of protrusions 108 having square or rectangular cross sections will produce depressions 110 having a square cross section. Alternatively, a series of protrusions having conical cross sections will produce depressions having a conical cross section.

As an alternative to the die assembly 100, the die assembly 70 of FIG. 4 or the die assembly 172 of FIG. 12 can be used to impart pressure concentrating features onto a lithium sheet or piece alone, before it is laminated to the current collector. This may be done before or after a larger lithium sheet is cut to be a lithium piece sized for lamination to a current collector.

Yet another alternative embodiment of a die assembly for imparting pressure concentrating features onto a lithium sheet or piece alone is shown in FIG. 22. Die assembly 126 is constructed to apply force to a workpiece through linear motion rather than rotary motion as in FIG. 21. Die assembly 126 includes a die base 128 and a pressure plate 130. Die base 128 has a plurality of protrusions 132, where there are many usable shapes of protrusions 132. For example, protrusions 132 may have a generally square cross section, as shown in FIG. 22, or may have a conical cross section, semi-spherical cross section, or other cross section. The protrusions 132 may have profiles similar to the ridges 142 of FIGS. 16-18, for example. Pressure plate 130 has a series of reliefs 150 configured to provide clearance for material that is deformed by protrusions 132 and is otherwise generally smooth or planar. Reliefs 150 may be configured to provide clearance with each individual protrusion 132 or may be configured to provide clearance to a region of protrusions 132 or all protrusions 132. When a sheet of lithium 134 is placed within die assembly 126 and force is applied to lithium sheet 134, the protrusions 132 on die base 128 tend to create a series of depressions 136 and protrusions 138 on both sides of the lithium sheet 134. These depressions 136 and protrusions 138 function in the same fashion as described above to promote effective contact between a lithium anode and a current collector, as well as to provide for microextrusion of the surface to expose fresh lithium material to the current collector.

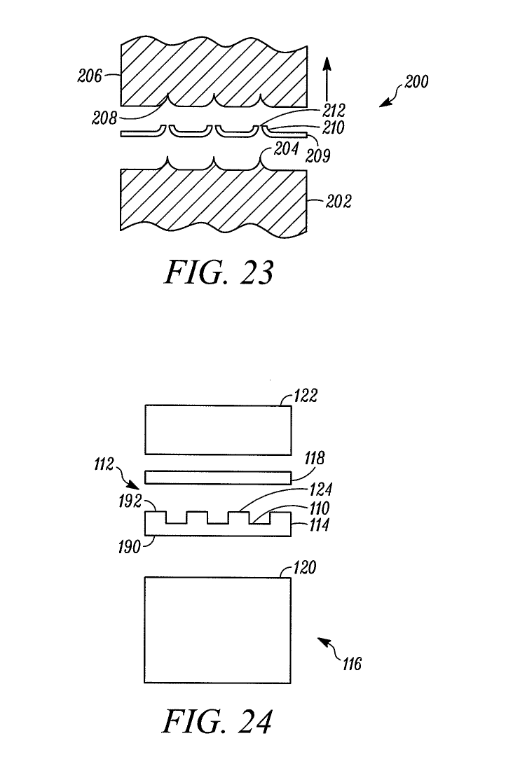

Another embodiment of a die assembly 200 for preprocessing a lithium sheet or piece is illustrated in FIG. 23, where die base 202 includes pin-shaped or extended conical-shaped protrusions 204. Pressure plate 206 includes reliefs 208 for accommodating the protrusions 204. In one embodiment, the protrusions 204 are pushed through a sheet 209 of lithium to create high spots 210 that are slightly necked, having holes 212 or partial holes in the lithium. These necked high spots 210 will be compressed during lamination of the lithium sheet to a current collector. When the lithium sheet 209 is laminated to a current collector, the high spots 210 are oriented to be facing the current collector. As pressure is applied to the die assembly 200, the lithium of high spots 210 and surrounding the holes 212 or partial holes will extrude into the holes 212, thus tending to expose unreacted lithium to the current collector. The high spots 210 create high pressure regions during lamination to a current collector, thus increasing the mechanical interaction between the layers.

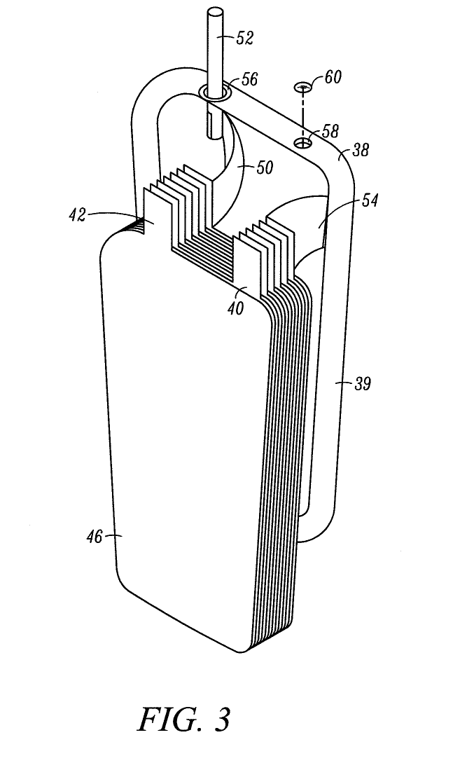

After a sheet or piece of lithium has been processed in a die assembly such as die assembly 72, 100, 126, 172 or 200 to form a structured surface, it is further processed in a customary manner to form an anode assembly. If a web or sheet of lithium was processed to form a structured surface, then the web or sheet of lithium is die-punched to form a lithium piece of the desired shape and profile for a battery. As illustrated in FIG. 24, this lithium piece 114 is then laminated to a current collector 118 using a die assembly 116 having a base 120 and a pressure plate 122. In FIG. 24, the lithium sheet 114 is illustrated as having one structured side 192 and one flat side 190. However, lithium sheet 114 may also have two sides that are structured, such as if formed by die assembly 126 or 200. The surfaces of base 120 and pressure plate 122 that contact lithium sheet 114 and nickel current collector 118, respectively, are generally flat or planar. Force is applied to die assembly 116 to bring base 120 and pressure plate 122 together and to apply a pressure to lithium sheet 114 and current collector 118. Force applied to lithium sheet 114 is borne only by protrusions 124, which have a relatively small overall surface area. For a given force applied by die assembly 116, this small surface area contact leads to relatively high contact pressures. In a similar manner as described above in connection with the embodiment depicted in FIG. 3, these relatively high contact pressures will result in a more effective lamination of the lithium sheet 114 to the current collector 118, particularly in the regions of protrusions 124. Sufficient force is applied to at least partially deform protrusions 124. Furthermore, the lithium of the protrusions 124 tends to extrude outward under pressure, into the depressions 110, causing the surface of the lithium of the protrusions 124 to break or smear and thereby expose fresh lithium to the surface of the current collector 118. In addition, the extrusion of lithium into the depressions 110 tends to prevent lithium from extruding beyond the outer profile of the current collector 118. This tends to reduce the likelihood of the anode assembly 112 from contacting the cathode 28 when the components are assembled into a battery.

The preceding description of the various aspects of the invention has been limited for clarity and ease of description to describing processing of the lithium anode 32 and features associated with the lithium anode 32. However, it will be appreciated that the invention is also fully applicable to processing of the current collector 34 and to features associated with current collector 34. Any processing step that has been described with reference to anode 32 is equally applicable to current collector 34 and is expected to produce similar benefits. Likewise, any feature that has been described with reference to anode 32 is equally applicable to current collector 34 and is expected to produce similar benefits. By way of example but without limitation, pressure concentrating features may be formed on current collector 34 instead of on anode 32.

Additional details and examples related to the components of a battery for an implantable medical device will now be described, with reference to FIGS. 1-3. Many embodiments of battery assembly 20 are usable, including variations in the number of repeating assemblies 22 that are used. In some embodiments, an additional anode assembly 24 is positioned proximate to the cathode 28 side of the last repeating arrangement 22.

The separator 26 is designed to allow ionic communication between anode assembly 24 and cathode 28 while preventing electrical contact between the two components. The separator 26 may be constructed from any of a number of materials. For example, in various embodiments, separator 26 is constructed from a microporous polypropylene membrane, a microporous polyethylene membrane, or a layer of polyethylene laminated between two layers of polypropylene. In one embodiment, a separator including polypropylene provides relatively high strength and toughness while the relatively low melt temperature of the polyethylene is advantageous in the event of a short circuit. During a short circuit, the elevated cell temperature causes the pores of separator 26 to melt together or “shut down,” which in turn reduces Li ion transport and causes the cell to cool down safely.

Many embodiments of lithium anode 32 and current collector 34 are usable. In various usable embodiments, the lithium anode 32 is about 0.001 to 0.05 inches (0.025 to 1.27 mm) thick, and in some embodiments, about 0.002 inch (0.05 mm) thick or 0.003 inch (0.08 mm) thick or 0.005 inch (0.127 mm) thick. In various usable embodiments, nickel current collector 34 is about 0.0001 to 0.005 inches (0.0025 to 0.127 mm) thick, and in some embodiments about 0.001 inch (0.025 mm) thick or 0.002 inch (0.05 mm) thick.

The anode assembly 24 also includes a tab 40 (as shown in FIG. 1) for making an electrical connection to the current collector 34. The pressed-together anode 32 and current collector 34 are then sealed within a microporous separator 27, or “baggie.” The separator 27 is constructed from material similar to the material of separator 26 and is configured to perform the same functions, namely, allowing ionic communication between anode assembly 24 and cathode 28 while preventing electrical contact between the two components and also melting during a short circuit to allow the cell to cool down safely. There are many usable techniques for sealing separator 27, such as heat sealing using thermal impulse sealing, ultrasonic sealing, laser sealing, heat or mechanical staking, or taping.

In various embodiments, the cathode includes at least one metal oxide, metal sulphide, metal selenide, metal halide or metal oxyhalide compound or their corresponding lithiated forms. The cathode 28 may include manganese, vanadium, silver, molybdenum, tungsten, cobalt, nickel, or chromium. The cathode may also include a main group compound such as carbon monofluoride or iodine. Other compositions of the cathode are within the scope of this disclosure. In one example, the cathode 28 is formed from a mixture that includes manganese dioxide (MnO₂) active material. In one embodiment, cathode 28 includes a substrate that is coated with slurry composed of an active material, which is then dried, calendared, and die cut to a final shape. According to various other embodiments, the cathode is made from compressed powder, dough and/or slurry.

In one embodiment, the cathode substrate is constructed from tabs 42 (as shown in FIG. 1) that are welded to an exposed layer. Many embodiments of tabs 42 and the exposed layer are usable. For example, the tabs and exposed layer may be constructed from a variety of materials, such as stainless steel, aluminum, or copper. In some embodiments, the exposed layer is a grid and in other embodiments the exposed layer is a uniform sheet. In another embodiment, the slurry is formed from a mixture of manganese dioxide active material with a carbon conductive diluent and a binder. Many embodiments of the binder are usable. One usable embodiment of the binder is PTFE.

The edges of the cathode 28 contain a sheared metal material, which is quite sharp and can easily puncture the plastic separator 26, 30. For this reason, the cathodes 28 are preferably constructed to be larger than the anode assemblies 24. Preferably, the cathodes 28 are larger than the anode assemblies 24 by at least an amount that represents the size of the sheared material region, plus the expected variability in size, plus a margin of safety. In various example embodiments, cathodes 28 are constructed to be 0.005 to 0.1 inches (0.12 to 2.5 mm) larger than anode assemblies 24, and about 0.02 inches (0.5 mm) larger than anode assemblies.

A plurality of repeating arrangements 22, and possibly one additional cathode 38, are assembled together into a stack 44. After stack 44 is assembled, the cathode tabs 42 which were welded to the cathode are laser welded to each other to create a parallel interconnect configuration, thereby forming a cathode stack. Furthermore, the anode tabs 40 are welded together to create a similar parallel interconnect configuration on the anode side, forming an anode stack. A stainless steel case 38 is provided to contain stack 44, the case 38 being formed from two halves 37, 39 that are configured to mate together to contain stack 44.

In the embodiment shown in FIG. 3, a ribbon wire 50 is provided to connect the welded cathode tabs 42 of the cathode stack to a pin 52 that passes through an opening in case 38. A second ribbon wire 54 is provided to connect the welded anode tabs 40 of the anode stack to the case 38. In some embodiments, a glass bushing and/or a ferrule 56 is provided where pin 52 passes through case 38 to provide support to pin 52 as well as to electrically insulate pin 52 from case 38. Once stack 44 is positioned in case halves 37, 39 and the ribbon wires 50, 54 are connected, the case halves 37, 39 are brought together and the seam is laser welded, forming a hermetic seal around case 38.

The case 38 may also include a fill opening 58, as shown in FIG. 3. Fill opening 58 is configured to allow the hermetically sealed case 38 to be filled with electrolyte. Many different usable electrolyte compositions are known to those of skill in the art. After thermal soak processing, the fill opening 58 is closed. Many techniques are usable to close fill opening 58 after filling with electrolyte. In one usable embodiment, a plug 60 is secured within opening 58 such as by welding. Other usable embodiments exist to close fill opening 58, such as driving a plug into opening 58, welding across opening 58, or placing a cover over opening 58. The battery 20 is then preferably subjected to a series of electrical tests to confirm that it operates properly. In one embodiment of battery 20, the cell is designed to deliver sufficient current to provide a desired service life in a typical implantable cardioverter defibrillator (ICD).

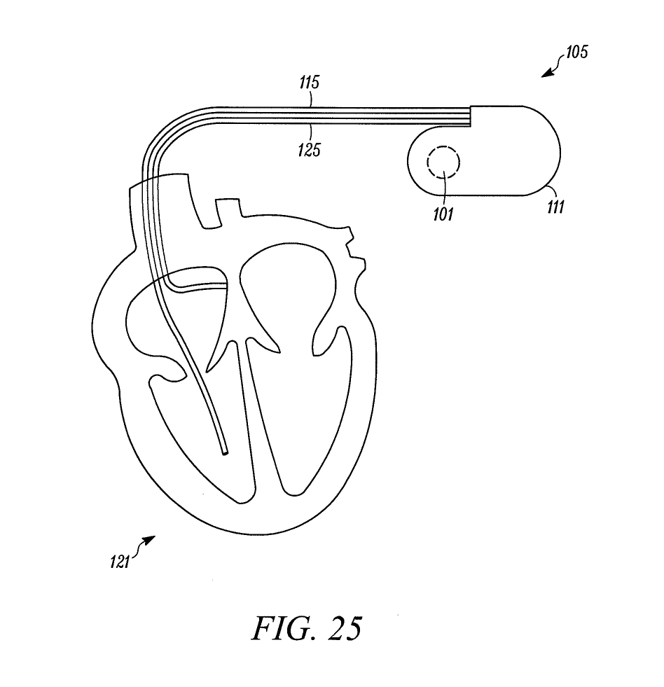

Referring now to FIG. 25, a lithium battery 101 is contained in an exemplary implantable device 105. In an example, the device 105 includes a lead assembly 115 extending into a heart 121 and a housing 111 containing a battery 101. In an example, the device also includes a second lead 125 that extends into the left side of the heart. In an example, the implantable device includes a defibrillator circuit, and the battery is configured to supply a high energy signal through the defibrillator circuit. The implantable medical device also includes circuitry configured to send and receive electrical impulses through the leads 115, 125. The lithium battery is configured to provide electrical power to the circuitry. After the lithium battery 101 is assembled in its battery housing, the implantable medical device 105 is assembled and includes the battery housing.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

What is claimed is:

1. A method comprising the steps of:
(i) providing a sheet of lithium and a sheet of substrate material;
(ii) pressing the sheet of lithium and the sheet of substrate material together in a die to form an electrode, the die having at least one surface that includes a plurality of force concentrating features configured to create regions of relatively higher pressure and regions of relatively lower pressure in at least one of the sheet of lithium and the sheet of substrate material;
(iii) assembling a battery including the electrode in a battery housing; and
(iv) assembling an implantable medical device including the battery housing.

2. The method of claim 1, wherein the force concentrating features comprise a plurality of one of a group consisting of depressions, protrusions, ridges and grooves.

3. The method of claim 2, wherein the plurality of force concentrating features are regularly spaced on the surface of the die.

4. The method of claim 2, wherein the force concentrating features comprise a plurality of depressions, wherein each depression is characterized by a generally round profile at the die surface.

5. The method of claim 2, wherein the force concentrating features comprise a plurality of protrusions, wherein the plurality of protrusions are characterized by a generally round profile at the die surface.

6. The method of claim 2, wherein each force concentrating feature is characterized by a width or diameter at the die surface of about 0.001 to 0.005 inches (0.025 to 0.127 mm).

7. The method of claim 2, wherein the force concentrating features comprise a plurality of grooves, wherein the plurality of grooves comprise a first series of generally parallel grooves and a second series of generally parallel grooves, where each individual groove of the first series of grooves is generally perpendicular to each individual groove of the second series of grooves.

8. The method of claim 2, wherein the force concentrating features comprise a plurality of ridges, wherein the plurality of ridges comprise a first series of generally parallel ridges and a second series of generally parallel ridges, where each individual ridge of the first series of ridges is generally perpendicular to each individual ridge of the second series of ridges.

9. The method of claim 1, wherein the step of pressing includes applying sufficient force to cause lithium that is free from lithium reaction byproducts to be present at an interface between the substrate material and the surface of the lithium.

10. The method of claim 1, wherein the step of pressing comprises pressing with sufficient force to cause indentations corresponding to the pressure concentrating features in one of the sheet of lithium and the sheet of substrate material.

11. The method of claim 10, wherein the indentations are characterized by a depth of about 0.0001 to 0.003 inches (0.0025 to 0.076 mm).

12. The method of claim 10, further including a step of applying sufficient force to a surface having indentations to at least partially deform the indentations.

13. The method of claim 1, wherein the substrate material comprises nickel or a nickel-containing material.

14. A method comprising the steps of:
(i) providing a sheet of lithium material and a substrate material;
(ii) applying force to the sheet of lithium and substrate material to form a plurality of protrusions on at least one of the sheet of lithium and substrate material;
(iii) pressing at least a portion of the sheet of lithium to the substrate material with sufficient force to at least partially deform the protrusions and form an electrode;
(iv) assembling a battery including the electrode in a battery housing; and
(v) assembling an implantable medical device including the battery housing.

15. The method of claim 14, wherein the step of applying force to a sheet of lithium and substrate material to form a plurality of protrusions comprises forming protrusions that have a generally conical, semi-spherical, pin-type or square cross-section.

16. The method of claim 14, wherein the step of applying force to a sheet of lithium and substrate material comprises passing the sheet of lithium through a nip formed by a first roller and a second roller, where at least one of the first and second rollers has a plurality of protrusion-forming features.

17. The method of claim 15, wherein the step of applying force to a sheet of lithium and substrate material comprises applying force to a die assembly, the die assembly having at least one surface having a plurality of protrusion-forming features.

18. The method of claim 14, further comprising the step of cutting the sheet of lithium to form the portion of the sheet of lithium before pressing the portion of the sheet of lithium to the substrate material.

19. The method of claim 14, wherein the step of pressing comprises applying force to a second die assembly having planar surfaces.

20. A tool for processing a sheet for use in an electrode of a battery, the tool comprising:
(i) a first rigid surface having a plurality of force concentrating features, the first rigid surface being configured to engage with a surface of the sheet;
(ii) a second rigid surface, the second rigid surface being positioned opposite to the first rigid surface; and
(iii) a mechanism to force the first and second surfaces together against at least the sheet with sufficient force that the plurality of force concentrating features will at least partially deform the sheet.

21. The tool of claim 20, wherein the first and second rigid surfaces are generally planar.

22. The tool of claim 20, wherein the first and second rigid surfaces are generally cylindrical.

23. The tool of claim 20, wherein the second rigid surface is generally smooth.

24. The tool of claim 20, wherein plurality of force concentrating features are protrusions, wherein the second rigid surface has one or more openings configured to provide clearance opposite to the protrusions of the first rigid surface when the first and second surfaces are pressed together, the second surface being configured to engage with at least a portion of the second surface of the sheet.

25. An implantable medical device comprising:
a housing;
at least one lead;
circuitry configured to send and receive electrical impulses through the lead; and
a lithium battery configured to provide electrical power to the circuitry, the battery comprising:
an electrode comprising a lamination formed from at least a sheet of lithium and a sheet of a substrate material, the lamination having one of a group consisting of:
a plurality of partially compressed protrusions on the sheet of lithium; and
a plurality of projections of the sheet of lithium into the surface of the sheet of substrate material.
